(19) United States
(12) Patent Application Publication
(10) Pub. No.: US 2011/0021569 A1
(43) Pub. Date: Jan. 27, 2011

(54) Piperidine compounds, a process for their preparation and pharmaceutical compositions containing them

(75) Inventors: Iuliana Botez, Houilles (FR); Jacques Lebreton, Nantes (FR); Pierre Lestage, Le Celle Saing Cloud (FR); Caroline Louis, Paris (FR); Monique Mathe, Nantes (FR); Daniel-Henri Caignard, Boisemont (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignees: LES LABORATOIRES SERVIER, UNIVERSITE, Suresnes Cedex (FR); UNIVERSITE DE NANTES, Nantex Cedex (FR)

(21) Appl. No.: 12/804,360

(22) Filed: Jul. 20, 2010

(30) Foreign Application Priority Data
Jul. 21, 2009 (FR) 09.03573

(51) Int. Cl. A61K 31/445; C07D 211/94; A61P 25/00; A61P 25/16; A61P 25/28; A61P 25/18

(52) U.S. Cl. 514/327; 546/242

(57) ABSTRACT

Compounds of formula (I):

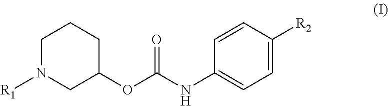

wherein:
R₁ represents a hydrogen atom or a methyl group,
R₂ represents a bromine atom, a fluorine atom or a trifluoromethyl group.

Medicinal products containing the same which are useful in treating cognitive disorders associated with pathologies of the central nervous system.

The present invention relates to new substituted piperidine compounds, to a process for their preparation, to pharmaceutical compositions containing them and also to the use thereof as facilitators of memory and cognition in the treatment of cognitive disorders associated with pathologies of the central nervous system.

Ageing of the population due to increased life expectancy has brought with it a major increase in cognitive disorders associated with normal cerebral ageing and with pathological cerebral ageing occurring in the course of neurodegenerative diseases such as, for example, Alzheimer's disease.

The majority of substances used today in treating cognitive disorders associated with ageing act by facilitating the central cholinergic systems—either directly, as in the case of acetylcholinesterase inhibitors (tacrine, donepezil) and cholinergic agonists (nefiracetam), or indirectly, as in the case of nootropic agents (piracetam, pramiracetam) and cerebral vasodilators (vinpocetine).

Besides their cognitive properties, substances acting directly on the central cholinergic systems often have hypothermic properties, which can be undesirable.

It has been therefore been especially valuable to synthesise new compounds that are capable of countering the cognitive disorders associated with ageing and/or of improving cognitive processes, without having hypothermic activity.

Substituted piperidine compounds described as products of synthesis and/or alkaloids are known from the literature (J. Chem. Soc., Perkin Trans. 1, 1991, 3, pp. 611-616; Heterocycles, 1985, 23(4), pp. 831-834; Can. J. Chem., 1996, 74(12), pp. 2444-2453).

The compounds of the present invention are new and have especially valuable properties from a pharmacological point of view.

The present invention relates more especially to compounds of formula (I):

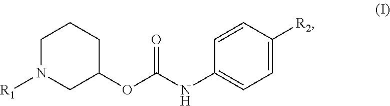

wherein:
R₁ represents a hydrogen atom or a methyl group,
R₂ represents a bromine atom, a fluorine atom or a trifluoromethyl group,
   to their enantiomers, and also to addition salts thereof with a pharmaceutically acceptable acid or base.

Among the pharmaceutically acceptable acids there may be mentioned, without implying any limitation, hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, oxalic acid etc.

Among the pharmaceutically acceptable bases there may be mentioned, without implying any limitation, sodium hydroxide, potassium hydroxide, triethylamine, tert-butylamine etc.

The R₁ group advantageously represents a methyl group.

Preference is given to the R₂ group being a bromine atom.

The invention relates preferably to compounds which are:
1-methyl-piperidin-3-yl (4-bromophenyl)carbamate,
(3S)-1-methyl-piperidin-3-yl (4-bromophenyl)carbamate,
(3R)-1-methyl-piperidin-3-yl (4-bromophenyl)carbamate.

The addition salts of preferred compounds of the invention with a pharmaceutically acceptable acid or base form an integral part of the invention.

The invention relates also to a process for the preparation of compounds of formula (I), characterised in that there is used as starting material the compound of formula (II):

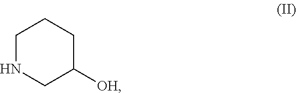

which is subjected to a step of protection of the nitrogen atom to yield the compound of formula (III):

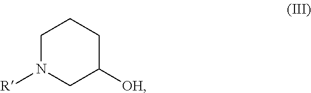

wherein R′ represents a protecting group for the amine function such as, for example, a tert-butyloxycarbonyl group,
which is then subjected to the action of a compound of formula (IV),

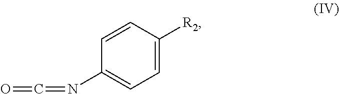

wherein R₂ is as defined for formula (I),
to yield the compound of formula (V):

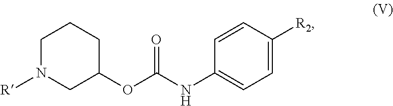

wherein R₂ and R′ are as defined hereinbefore,
which is then subjected to a reaction deprotecting the amine function, for example in the presence of trifluoroacetic acid, to yield the compounds of formula (I/a), a particular case of the compounds of formula (I),

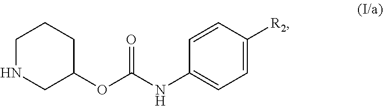

wherein R₂ is as defined hereinbefore,
which may optionally be subjected to a methylation reaction to yield the compounds of formula (I/b), a particular case of the compounds of formula (I),

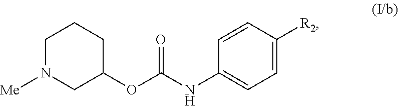

wherein R₂ is as defined hereinbefore,
a variant for the preparation of the compound of formula (I/b) consisting of using as starting material the compound of formula (VI):

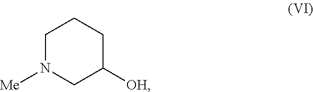

which is subjected to the action of a compound of formula (IV),
which compounds of formulae (I/a) and (I/b), which constitute the entirety of the compounds of formula (I), may then be purified according to a customary separation technique, which are converted, if desired, into addition salts thereof with a pharmaceutically acceptable acid or base and the enantiomers of which may be separated on a chiral column, according to a customary separation technique.

The compounds of formulae (II), (IV) and (VI) are commercially available or readily accessible to the person skilled in the art by means of customary chemical reactions or chemical reactions described in the literature.

Besides the fact that they are new, the compounds of the present invention have properties facilitating cognitive processes, making them of use in the treatment of cognitive deficiencies associated with cerebral ageing and with neurodegenerative diseases such as Alzheimer's disease, Parkinson's disease, Pick's disease, Korsakoff's disease, vascular dementias, frontal and sub-cortical dementias and also schizophrenia.

The invention relates also to pharmaceutical compositions comprising as active ingredient at least one compound of formula (I) together with one or more suitable, inert, non-toxic excipients. Among the pharmaceutical compositions according to the invention there may be mentioned more especially those that are suitable for oral, parenteral (intravenous or subcutaneous) or nasal administration, tablets or dragées, sublingual tablets, capsules, lozenges, suppositories, creams, ointments, dermal gels, injectable preparations, drinkable suspensions etc.

The useful dosage will depend on the nature and severity of the disorder, the administration route and also the age and weight of the patient and ranges from 0.01 mg to 1 g per day in one or more administrations.

The Examples that follow illustrate the invention but do not limit it in any way.

The structures of the compounds described in the Examples were determined by customary spectrophotometric techniques (infrared, NMR, mass spectrometry).

### EXAMPLE 1

### 1-Methylpiperidin-3-yl (4-bromophenyl)carbamate

para-Bromophenyl isocyanate (25.4 mmol) is added, at ambient temperature, to a stirred solution of 3-hydroxy-1-methylpiperidine (21.2 mmol) in anhydrous toluene (50 mL) under an atmosphere of argon. The reaction mixture is stirred for 24 hours at 60° C. After filtering off over a frit, the solid material is washed with dichloromethane. The filtrate is evaporated to dryness under reduced pressure and is then taken up in dichloromethane. The organic phase is washed with saturated aqueous NaHCO₃ solution, dried over MgSO₄, filtered and then evaporated under reduced pressure. The yellow solid obtained is triturated in diisopropyl ether (60 ml). After filtration, the title product is obtained in the form of a white powder.

Melting point: 127° C.

### EXAMPLE 2

### (3R)-1-Methylpiperidin-3-yl (4-bromophenyl)carbamate

The R enantiomer was obtained by separation of the racemic mixture obtained in Example 1. Separation was carried out on a Chiralpak AS column at ambient temperature with UV detection at 275 nm, using a mixture of acetonitrile/diethylamine 100/0.1 as eluant.

Optical purity: >99%

Optical rotation: [α]^{20° C.}_{589 nm}=+13.57° (c=0.6 MeOH)

### Elemental Microanalysis:

| | C | H | N | Br |
|---|---|---|---|---|
| theoretical % | 49.86 | 5.47 | 8.94 | 25.51 |
| experimental % | 49.79 | 5.48 | 9.22 | 26.30 |

### EXAMPLE 3

### (3S)-1-Methylpiperidin-3-yl (4-bromophenyl)carbamate

The S enantiomer was obtained under the same conditions as those described in Example 2, starting from the racemic mixture obtained in Example 1.

Optical purity: >99%

Optical rotation: [α]^{20° C.}₅₈₉ₙₘ=−13.39° (c=1.0 MeOH)

### Elemental Microanalysis:

| | C | H | N | Br |
|---|---|---|---|---|
| theoretical % | 49.86 | 5.47 | 8.94 | 25.51 |
| experimental % | 49.96 | 5.50 | 9.07 | 25.98 |

### EXAMPLE 4

### 1-Methylpiperidin-3-yl (4-fluorophenyl)carbamate

para-Fluorophenyl isocyanate (44.0 mmol) is added, at ambient temperature, to a stirred solution of 3-hydroxy-1-methylpiperidine (34.7 mmol) in anhydrous toluene (50 mL) under an atmosphere of argon. The reaction mixture is stirred for 48 hours at 60° C. Having been filtered off over a frit, the solid material is washed with dichloromethane. The filtrate is evaporated to dryness under reduced pressure and is then taken up in dichloromethane. The organic phase is washed with saturated aqueous NaHCO₃ solution, dried over MgSO₄, filtered and then evaporated under reduced pressure. The orange oil obtained is purified by chromatography on silica gel using a mixture of dichloromethane/methanol (gradient from 99/1 to 95/5) as eluant. The white solid obtained is recrystallised from a minimum of diisopropyl ether to yield the title product in the form of a white powder.

Melting point: 97-98° C.

### EXAMPLE 5

### (3R)-1-Methylpiperidin-3-yl (4-fluorophenyl)carbamate

The R enantiomer was obtained by separation on a chiral preparative column.

### Elemental Microanalysis:

| | C | H | N |
|---|---|---|---|
| theoretical % | 61.89 | 6.79 | 11.10 |
| experimental % | 62.06 | 6.98 | 10.96 |

### EXAMPLE 6

### (3S)-1-Methylpiperidin-3-yl (4-fluorophenyl)carbamate

The S enantiomer was obtained by separation on a chiral preparative column.

### EXAMPLE 7

### 1-Methylpiperidin-3-yl (4-trifluoromethylphenyl)carbamate

para-Trifluoromethylphenyl isocyanate (7 mmol) is added, at ambient temperature, to a stirred solution of 3-hydroxy-1-methylpiperidine (4.8 mmol) in anhydrous toluene (10 mL) under an atmosphere of argon. The reaction mixture is stirred for 17 hours at 55° C. The crude reaction product is filtered over a frit; the solid material is then washed with dichloromethane. The filtrate is evaporated to dryness under reduced pressure and is then taken up in dichloromethane. The organic phase is washed with saturated aqueous NaHCO₃ solution, dried over MgSO₄, filtered and then evaporated under reduced pressure. The solid material is purified by chromatography on a column of silica gel using a mixture of dichloromethane/methanol (gradient from 98/2 to 95/5) as eluant to yield the title product in the form of a white powder.

Melting point: 119-120° C.

### EXAMPLE 8

### (3S)-1-Methylpiperidin-3-yl (4-trifluoromethylphenyl)carbamate

The S enantiomer was obtained by separation on a chiral preparative column.

Optical rotation: [α]^{20° C.}_{589 nm}=−12.44° (c=0.6 MeOH)

### Elemental Microanalysis:

| | C | H | N |
|---|---|---|---|
| theoretical % | 55.63 | 5.67 | 9.27 |
| experimental % | 55.45 | 6.04 | 9.26 |

### EXAMPLE 9

### (3R)-1-Methylpiperidin-3-yl 4-(trifluoromethylphenyl)carbamate

The R enantiomer was obtained by separation on a chiral preparative column.

Optical rotation: [α]^{20° C.}_{589 nm}=+13.96° (c=0.7 MeOH)

### Elemental Microanalysis:

| | C | H | N |
|---|---|---|---|
| theoretical % | 55.63 | 5.67 | 9.27 |
| experimental % | 55.67 | 6.26 | 9.32 |

### EXAMPLE 10

### Piperidin-3-yl (4-trifluoromethylphenyl)carbamate

### Step A: tert-Butyl 3-hydroxypiperidine-1-carboxylate

To a stirred solution of 3-hydroxypiperidine (39.6 mmol) in water (200 mL) there are added NaHCO₃ (119 mmol) and then di-tent-butyl carbonate (47.5 mmol) at ambient temperature. The reaction mixture is stirred for 3 days at ambient temperature and then the aqueous phase is extracted three times with dichloromethane. The organic phases are combined, dried over MgSO₄, filtered and then evaporated under reduced pressure. The colourless oil obtained is left to stand uncovered and at ambient temperature for 4 days. The crystals that are formed are then ground into a powder and placed under a vane pump for 24 hours. The title product is obtained in the form of white crystals without further purification.

### Step B: tert-Butyl 3-{[(4-trifluoromethylphenyl)amino]carbonyl}oxypiperidine-1-carboxylate

para-Trifluoromethylphenyl isocyanate (13.3 mmol) is added, at ambient temperature, to a stirred solution of the compound obtained in the Step above (8.4 mmol) in anhydrous toluene (30 mL) under an atmosphere of argon. The reaction mixture is stirred for 48 hours at 55° C. After filtration over a frit, the solid material is washed with copious amounts of dichloromethane. The filtrate is evaporated to dryness under reduced pressure to yield the title product in the form of a white powder.

### Step C: Piperidin-3-yl (4-trifluoromethylphenyl)carbamate

Trifluoroacetic acid (10.2 ml) is added to a stirred solution of the compound obtained in the Step above (6.9 mmol) in anhydrous dichloromethane (24 mL), under an atmosphere of argon, in an ice bath. The reaction mixture is stirred for 1 hour at ambient temperature. The crude reaction product is poured into 2M aqueous sodium hydroxide solution (100 ml) cooled by an ice bath. The basic aqueous phase is extracted twice with dichloromethane. The organic phases are combined, washed with saturated aqueous NaCl solution, dried over MgSO₄, filtered and then evaporated under reduced pressure to yield the title product in the form of a powder.

Melting point: 150-151° C.

### EXAMPLE 11

### (3S)-Piperidin-3-yl (4-trifluoromethylphenyl)carbamate

The S enantiomer was obtained by separation of the racemic mixture obtained in Example 10. Separation was carried out on a Chiralpak AD column at ambient temperature with UV detection at 245 nm, using a mixture of acetonitrile/diethylamine 100/0.1 as eluant.

Optical purity: >99%

Optical rotation: [α]^{20° C.}_{589 nm}=−14.04° (c=0.9 MeOH)

### Elemental Microanalysis:

| | C | H | N |
|---|---|---|---|
| theoretical % | 54.17 | 5.24 | 9.72 |
| experimental % | 54.20 | 5.38 | 9.62 |

### EXAMPLE 12

### (3R)-Piperidin-3-yl (4-trifluoromethylphenyl)carbamate

The R enantiomer was obtained under the same conditions as those described in Example 11 starting from the racemic mixture obtained in Example 10.

Optical purity: 99%

Optical rotation: [α]^{20° C.}_{589 nm}=+13.43° (c=1.2 MeOH)

### Elemental Microanalysis:

| | C | H | N |
|---|---|---|---|
| theoretical % | 54.17 | 5.24 | 9.72 |
| experimental % | 54.08 | 5.44 | 9.61 |

### EXAMPLE 13

### Piperidin-3-yl(4-bromophenyl)carbamate

### Step A: tert-Butyl 3-{[(4-bromophenyl)amino]carbonyl}oxypiperidine-1-carboxylate

para-Bromophenyl isocyanate (5.05 mmol) is added, at ambient temperature, to a stirred solution of the compound obtained in Step A of Example 10 (2.49 mmol) in anhydrous toluene (10 mL) under an atmosphere of argon. The reaction mixture is stirred for 72 hours at 60° C. The crude reaction product is filtered over a frit, evaporated to dryness under reduced pressure and then purified by chromatography on silica gel using a mixture of dichloromethane/diethyl ether (gradient from 100/0 to 90/10) as eluant. The title product is obtained in the form of a white powder.

### Step B: Piperidin-3-yl (4-bromophenyl)carbamate

Trifluoroacetic acid (13 ml) is added to a stirred solution of the compound of the Step above (10 mmol) in anhydrous dichloromethane (50 mL), under an atmosphere of argon, in an ice bath. The reaction mixture is stirred for 2 hours at ambient temperature. The crude reaction product is poured into saturated K₂CO₃ solution at 0° C. The aqueous phase is extracted twice with dichloromethane. The organic phases are combined, washed with 2N sodium hydroxide solution and then with saturated NaCl solution, dried over MgSO₄, filtered and then evaporated to yield the title product in the form of a white solid.

Melting point: 169-170° C.

### EXAMPLE 14

### (3S)-Piperidin-3-yl (4-bromophenyl)carbamate

The S enantiomer was obtained by separation on a chiral preparative column.

### Elemental Microanalysis:

| | C | H | N | Br |
|---|---|---|---|---|
| theoretical % | 48.18 | 5.05 | 9.36 | 26.71 |
| experimental % | 48.86 | 5.14 | 8.93 | 25.35 |

### EXAMPLE 15

### (3R)-Piperidin-3-yl(4-bromophenyl)carbamate

The R enantiomer was obtained by separation on a chiral preparative column.

### EXAMPLE 16

### Piperidin-3-yl(4-fluorophenyl)carbamate

### Step A: tert-Butyl 3-{[(4-fluorophenyl)amino]carbonyl}oxypiperidine-1-carboxylate

para-Fluorophenyl isocyanate (18 mmol) is added, at ambient temperature, to a stirred solution of the compound obtained in Step A of Example 10 (15 mmol) in anhydrous toluene (50 mL) under an atmosphere of argon. The reaction mixture is stirred for 72 hours at 60° C. Having been filtered over a frit, the solid material is taken up in dichloromethane (40 ml) and then is filtered over a frit again. The organic phase is washed with saturated aqueous NaHCO₃ solution, dried over MgSO₄, filtered and then evaporated under reduced pressure. The title product is obtained in the form of a white powder.

### Step B: Piperidin-3-yl (4-fluorophenyl)carbamate

Trifluoroacetic acid (23 ml) is added to a stirred solution of the compound obtained in the Step above (18.4 mmol) in anhydrous dichloromethane (100 mL), under an atmosphere of argon, in an ice bath. The reaction mixture is stirred for 3 hours at ambient temperature. The crude reaction product is poured into 2M aqueous sodium hydroxide solution (100 ml) placed in an ice bath. The aqueous phase is extracted twice with dichloromethane. The organic phases are combined, washed with saturated aqueous NaCl solution, dried over MgSO₄, filtered and then evaporated under reduced pressure. The crude product is purified by chromatography on an alumina column using a mixture of dichloromethane/methanol (gradient from 98/2 to 95/5) as eluant to yield the title product in the form of a white powder.

Melting point: 147-148° C.

### EXAMPLE 17

### (3R)-Piperidin-3-yl (4-fluorophenyl)carbamate

The R enantiomer was obtained by separation on a chiral preparative column.

### EXAMPLE 18

### (3S)-Piperidin-3-yl (4-fluorophenyl)carbamate

The S enantiomer was obtained by separation on a chiral preparative column.

### Pharmacological Study

### EXAMPLE A

### Study of Acute Toxicity and Hypothermic Effects

The acute toxicity was evaluated after oral administration to groups of 5 mice (26±2 grams). The animals were observed at regular intervals in the course of the first day and daily during the two weeks following treatment.

The results show that the compounds of the present invention, up to the maximum tested dose of 100 mg/kg per os, do not bring about any undesirable effects or changes to temperature.

### EXAMPLE B

### Social Recognition in the Wistar Rat

Initially described in 1982 by Thor and Holloway (J. Comp. Physiol., 1982, 96, 1000-1006), the social recognition test has subsequently been proposed by various authors (Dantzer et al., Psychopharmacology, 1987, 91, 363-368; Perio et al., Psychopharmacology, 1989, 97, 262-268) for studying the mnemocognitive effects of new compounds. The test is based on the natural expression of the olfactory memory of the rat and its natural tendency to forget and allows evaluation of memorisation, by recognition of a young congeneric animal, by an adult rat. A young rat (21 days), taken at random, is placed for 5 minutes in the cage housing an adult rat. With the aid of a video device, the experimenter observes the social recognition behaviour of the adult rat and measures its overall duration. The young rat is then removed from the adult rat's cage and is placed in its own cage until the second introduction. The adult rat is then given the compound under test and, after 2 hours, is again brought into the presence (5 minutes) of the young rat. The social recognition behaviour is then observed again and its duration measured. The assessment criterion is the difference (T₂−T₁), expressed in seconds, between the “recognition” times of the 2 encounters.

The results obtained show a difference (T₂−T₁) between (−19) and (−40) seconds inclusive for doses ranging from 0.3 to 3 mg/kg. These results show that, at a low dose, memorisation is very substantially increased.

### EXAMPLE C

### Object Recognition in the Wistar Rat

The object recognition test in the Wistar rat was initially developed by Ennaceur and Delacour (Behav. Brain Res., 1988, 31, 47-59). This test is based on the spontaneous exploratory activity of the animal and has the characteristics of episodic memory in humans. This memory test is sensitive to ageing (Scali et al., Eur. J. Pharmacol., 1997, 325, 173-180) and to cholinergic dysfunctions (Bartolini et al., Pharm. Biochem. Behav. 1996, 53(2), 277-283) and is based on the differences in the exploration of 2 objects of fairly similar shape—one familiar, the other new. Prior to the test, the animals are habituated to the environment (an enclosure without an object). In the course of a first session, the rats are placed (3 minutes) in the enclosure, in which there are 2 identical objects. The duration of exploration is measured for each object. In the course of the second session (3 minutes), 24 hours later, 1 of the 2 objects is replaced by a new object. The duration of exploration is measured for each object. The assessment criterion is the difference, Delta, expressed in seconds, between the exploration times for the new object and for the familiar object in the course of the second session. The control animals, previously treated with the carrier by the oral route 60 minutes before each session, explore the familiar object and the new object in an identical manner, which indicates that the object introduced earlier has been forgotten. Animals treated with a compound that facilitates mnemocognition preferentially explore the new object, which indicates that the object introduced earlier has been remembered.

The results obtained show a difference, Delta, of between 6 and 12 seconds, inclusive, for doses ranging from 0.3 to 3 mg/kg. These results show that, at a very low dose, memorisation is substantially increased.

### EXAMPLE D

### Pharmaceutical Composition

| | |
|---|---|
| Formula for the preparation of 1000 tablets each containing | 10 g |
| 10 mg of (3S)-1-methyl-piperidin-3-yl (4- | |
| bromophenyl)carbamate (Example 3) | |
| Hydroxypropylcellulose | 2 g |
| Wheat starch | 10 g |
| Lactose | 100 g |
| Magnesium stearate | 3 g |
| Talc | 3 g |

1. A compound selected from those of formula (I): 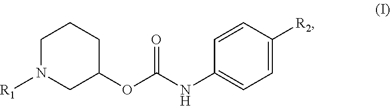
wherein:
R₁ represents a hydrogen atom or a methyl group,
R₂ represents a bromine atom, a fluorine atom or a trifluoromethyl group,
its enantiomers, and addition salts thereof with a pharmaceutically acceptable acid or base.

2. The compound of claim 1, wherein R₁ represents methyl.

3. The compound of claim 1, wherein R₂ represents a bromine atom.

4. The compound of claim 1, which is selected from 1-methyl-piperidin-3-yl (4-bromophenyl)carbamate, its enantiomers, and addition salts with a pharmaceutically acceptable acid or base.

5. The compound of claim 1, which is selected from (3S)-1-methyl-piperidin-3-yl (4-bromophenyl)carbamate and addition salts with a pharmaceutically acceptable acid or base.

6. The compound of claim 1, which is selected from (3R)-1-methyl-piperidin-3-yl (4-bromophenyl)carbamate and addition salts with a pharmaceutically acceptable acid or base.

7. A pharmaceutical composition comprising at least one compound of claim 1 or an addition salt thereof with a pharmaceutically acceptable acid or base in combination with one or more pharmaceutically acceptable excipients.

8. A method of treating deficiencies of memory associated with cerebral aging, neurodegenerative diseases, including Alzheimer's disease, Parkinson's disease, Pick's disease, Korsakoff's disease, vascular dementias, frontal and sub-cortical dementias, and schizophrenia in a subject in need thereof comprising administration of an effective amount of a compound of claim 1.
